(19) United States
(12) Patent Application Publication
(10) Pub. No.: US 2025/0049314 A1
(43) Pub. Date: Feb. 13, 2025

(54) METHOD AND SYSTEM FOR ASSESSING A CONDITION OF A PATIENT'S EYE

(71) Applicant: LIGHTX INNOVATIONS INC., Québec (CA)

(72) Inventors: Jean-Mathieu GAGNON, Québec (CA); Sébastien GAGNE, Québec (CA); Mathieu CHAMPAGNE, Québec (CA)

(21) Appl. No.: 18/719,938

(22) PCT Filed: Dec. 15, 2022
(86) PCT No.: PCT/CA2022/051833

§ 371 Date: Jun. 14, 2024

Related U.S. Application Data
(60) Provisional application No. 63/290,120, filed on Dec. 16, 2021.

(51) Int. Cl. A61B 3/10; A61B 3/00; A61B 3/135; A61B 3/14

(52) U.S. Cl. CPC A61B 3/1005; A61B 3/0025; A61B 3/135; A61B 3/14

(57) ABSTRACT

There is described a method for assessing a condition of a patient's eye. The method generally has: illuminating the patient's eye with a slit illumination beam from a first viewpoint; during said illuminating, imaging the patient's eye from a second viewpoint different from the first viewpoint, said imaging including generating a first image showing a first line element indicative of a reflection of the slit illumination beam on an iris of the patient's eye and a second line element indicative of a reflection of the slit illumination beam within a cornea of the patient's eye; fitting first and second lines to the first and second line elements; identifying a first intersection thereof; determining an angle value indicative of an angle between the first and second lines at the first intersection; and assessing the condition of the patient's eye based on the angle value.

### FIELD

The improvements generally relate to eye examination and more specifically relates to eye examination using slit lamps.

### BACKGROUND

A slit illuminator is an instrument having an illumination source that can be shaped as a thin strip of light and shined into a patient's eye, and a microscope for observing the illuminated eye for examination purposes. Slit lamps are generally operated by optometrists, ophthalmologist and other eye care professionals as they generally require a high level of training to precisely illuminate some specific parts of the eye such as the iris and cornea. In some circumstances, it can be desired to manipulate the slit illumination beam in order to allow some kind of qualitative appreciation of an angle formed between the iris and the cornea of the patient's eye using for instance a technique referred to as the Van Herick technique. This technique involves a slit illumination beam shined onto a periphery of the cornea at an angle of about 60° relative to the sagittal place of the patient. By observing the illuminated eye using the microscope, the eye care professional can evaluate that angle by qualitatively estimating a distance spacing the illuminated region of the cornea from the illuminated region of the iris of the patient's eye. Although the Van Herick technique is satisfactory to a certain degree, there remains room for improvement.

### SUMMARY

In accordance with a first aspect of the present disclosure, there is provided a method of assessing a condition of a patient's eye, the method comprising: using a slit illuminator, illuminating the patient's eye with a first slit illumination beam from a first viewpoint; using a camera and during said illuminating, imaging the patient's eye from a second viewpoint different from the first viewpoint, said imaging including generating a first image showing a first line element indicative of a reflection of the first slit illumination beam on an iris of the patient's eye and a second line element indicative of a reflection of the first slit illumination beam within a cornea of the patient's eye; and using a controller, fitting first and second lines to a respective one of the first and second line elements in the first image, identifying a first intersection of the first and second lines; determining a first angle value indicative of an angle formed between the first and second lines at the first intersection; and assessing the condition of the patient's eye based on the first angle value.

Further in accordance with the first aspect of the present disclosure, the first slit illumination beam can for example be directly focused on the patient's eye.

Still further in accordance with the first aspect of the present disclosure, the first line can for example be one of curved and linear, and the second line can for example be linear.

Still further in accordance with the first aspect of the present disclosure, the method can for example further comprise identifying a second intersection of the first and second lines different from the first intersection and determining a second angle value indicative of an angle formed between the first and second lines at the second intersection.

Still further in accordance with the first aspect of the present disclosure, the method can for example further comprise illuminating the patient's eye with a second slit illumination beam having a third viewpoint different from the first viewpoint, and imaging the patient's eye during said illuminating with the second slit illumination beam, said imaging generating a second image showing a third line element indicative of a reflection of the second slit illumination beam on the iris of the patient's eye and a fourth line element indicative of a reflection of the second slit illumination beam within the cornea of the patient's eye, the method further comprising repeating said fitting, said identifying and said determining for said second image, thereby outputting a second angle value on which said assessing is further based.

Still further in accordance with the first aspect of the present disclosure, the first slit illumination beam can for example have a first orientation with respect to the slit illuminator, the method further comprising illuminating the patient's eye with a second slit illumination beam having a second orientation being different from the first orientation, and imaging the patient's eye during said illuminating with the second slit illumination beam, said imaging generating a second image showing a third line element indicative of a reflection of the second slit illumination beam on the iris of the patient's eye and a fourth line element indicative of a reflection of the second slit illumination beam within the cornea of the patient's eye, the method further comprising repeating said fitting, said identifying and said determining for said second image, thereby outputting a second angle value on which said assessing is further based.

Still further in accordance with the first aspect of the present disclosure, the method can for example further comprise determining a thickness of the cornea based on a thickness of the second line element.

Still further in accordance with the first aspect of the present disclosure, the method can for example further comprise generating at least one of an iris three-dimensional (3D) model and a cornea 3D model based at least on the first angle value.

Still further in accordance with the first aspect of the present disclosure, said assessing can for example include matching the first angle value to the condition of the patient's eye based on reference data associating reference angle values to corresponding reference eye conditions.

Still further in accordance with the first aspect of the present disclosure, the reference angle values can for example originate from reference measurements performed at the first and second viewpoints.

Still further in accordance with the first aspect of the present disclosure, the method can for example further comprise determining a pixel count indicative of a number of pixels extending between the first and second lines, wherein said assessing is further based on said pixel count.

Still further in accordance with the first aspect of the present disclosure, the controller can for example have a trained engine performing at least one of said fitting, said identifying, said determining and said assessing.

In accordance with a second aspect of the present disclosure, there is provided a system for assessing a condition of a patient's eye, the system comprising: a frame; a slit illuminator mounted to the frame and having a first viewpoint, the slit illuminator being configured for illuminating the patient's eye with a slit illumination beam; a camera mounted to the frame and having a second viewpoint different from the first viewpoint, the camera being configured for imaging the patient's eye during said illuminating, said camera generating a first image showing a first line element indicative of a reflection of the slit illumination beam on an iris of the patient's eye and a second line element indicative of a reflection of the slit illumination beam within a cornea of the patient's eye; and a controller communicatively coupled to the camera, the controller having a processor and a memory having stored thereon instructions that when executed by the processor perform the steps of: fitting first and second lines to a respective one of the first and second line elements in the first image, identifying a first intersection of the first and second lines; determining a first angle value indicative of an angle formed between the first and second lines at the first intersection; and assessing the condition of the patient's eye based on the first angle value.

Further in accordance with the second aspect of the present disclosure, the slit illuminator can for example be movably mounted to the frame via a first encoding device, the first encoding device monitoring the first viewpoint of the slit illuminator with respect to the patient's eye when said first image is generated, and generating a signal indicative of the first viewpoint.

Still further in accordance with the second aspect of the present disclosure, the first encoding device can for example be further configured for monitoring an orientation of the slit illumination beam with respect to the frame, and generating a signal indicative of an orientation angle of the slit illumination beam when said first image is generated.

Still further in accordance with the second aspect of the present disclosure, the camera can for example be movably mounted to the frame via a second encoding device, the second encoding device monitoring the second viewpoint of the camera with respect to the patient's eye when said first image is generated, and generating a signal indicative of the second viewpoint.

Still further in accordance with the second aspect of the present disclosure, said assessing can for example further include matching the first angle value to the condition of the patient's eye based on reference data.

Still further in accordance with the second aspect of the present disclosure, the reference data can for example include a plurality of first angle values associated to a corresponding plurality of conditions of the eye for at least the first and second viewpoints.

Still further in accordance with the second aspect of the present disclosure, the system can for example further comprise determining a pixel count indicative of a number of pixels extending between the first and second lines, wherein said assessing is further based on said pixel count.

Still further in accordance with the second aspect of the present disclosure, the controller can for example comprise a trained engine performing at least one of said identifying, determining and assessing.

It is noted that the term “line element” is meant to encompass any elongated or line-like shapes which can be either linear or curved and which can have a given thickness extending perpendicularly to a length thereof. Similarly, the term “line(s)” is meant is to encompass lines that are either linear or curved.

Many further features and combinations thereof concerning the present improvements will appear to those skilled in the art following a reading of the instant disclosure.

### DESCRIPTION OF THE FIGURES

In the figures,

FIG. 1 is a view of an example of a system for assessing a condition of a patient's eye, shown with a slit illuminator, a camera and a controller, in accordance with one or more embodiments;

FIG. 1A is a top plan view the patient's eye being examined using the system of FIG. 1, taken along section 1A-1A of FIG. 1, in accordance with one or more embodiments;

FIG. 2 is an image generated by the camera of the system of FIG. 1, showing the patient's eye being illuminated with a slit illumination beam, in accordance with one or more embodiments;

FIG. 3 is a schematic view of an example of a computing device of the controller of FIG. 1, in accordance with one or more embodiments;

FIG. 4 is a schematic view of an example of a software application of the controller of FIG. 1, in accordance with one or more embodiments;

FIG. 5 is a schematic view of training images being associated with truth data to train the software application of FIG. 4, in accordance with one or more embodiments;

FIG. 6A is an image of a patient's eye being illuminated with a first slit illumination beam of a first orientation, in accordance with one or more embodiments;

FIG. 6B is an image of a patient's eye being illuminated with a second slit illumination beam of a second orientation different from the first orientation, in accordance with one or more embodiments;

FIG. 7 is a top plan view of another example of a system for assessing a condition of a patient's eye, shown with two spaced-apart cameras simultaneously imaging a patient's eye from different viewpoints, in accordance with one more embodiments;

FIGS. 8A and 8B are images of the patient's eye captured using the system of FIG. 7, in accordance with one or more embodiments; and

FIG. 9 is a flow chart of an example of a method of assessing a condition of a patient's eye, in accordance with one or more embodiments.

### DETAILED DESCRIPTION

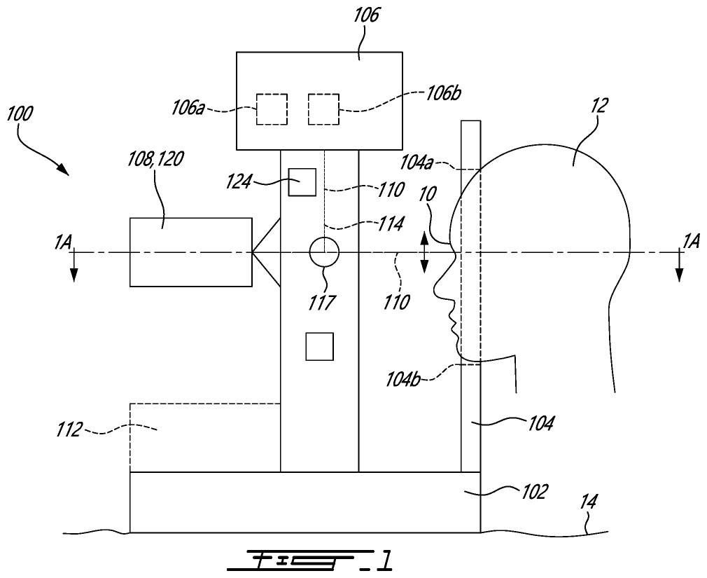

FIG. 1 shows an example of a system 100 for assessing a condition of an eye 10 of a patient 12 (“the patient's eye 10”). As depicted, the system 100 has a frame 102 to which are mounted a face receiving member 104, a slit illuminator 106, and a camera 108.

As shown in this example, the face receiving member 104 receives a face of the patient 12 during examination. The face receiving member 104 can be supported or fixed to a surface 14 such as a table, depending on the embodiment. The face receiving member 104 can have a forehead support 104a and/or a chin support 104b to comfortably receive the face of the patient 12.

It is intended that the slit illuminator 106 emits a slit illumination beam 110 towards the face received in the face receiving member 104 during the examination, and more specifically towards the eye 10 of the patient 12. The slit illuminator 106 is operable to illuminate the eye 10 of the patient 12 in one or more of different illumination patterns as can be expected from any type of existing slit lamp. For instance, in some embodiments, the slit illumination beam 110 is directly focused on the patient's eye in a direct and focal manner. The slit illuminator 106 can include a slit illuminator 106a and a background illuminator 106b in some embodiments. The slit illuminator 106 can be positioned above the head of the patient 12, such as in Haag Streit type slit lamps, or below the head of the patient 12, such as in Zeiss type slit lamps. As such, the illumination path 114 can be redirected from a substantially vertical path to a substantially horizontal path via one or more mirrors 117. The slit illumination beam can have a vertically-, obliquely- or horizontally-oriented slit, depending on the embodiment. Depending on the embodiment, the slit illuminator can have a light source such as a lamp, laser, grid-based projector, etc., as found suitable.

In some embodiments, the slit illuminator 106 is configured to be movable relative to the face of the patient. For instance, the slit illuminator 106 may be mechanically connected to the frame 102 via an articulated arm, or any other suitable type of actuator. In such embodiments, the slit illuminator 106 can be moved in any given coordinate system x, y, z relative to the eye of the patient. Accordingly, by moving the slit illuminator 106 during examination, one or more images of the eye 10 of the patient 12 can be taken under illumination from different viewpoints. One or more of the patient's eye 10 can be taken under illumination from different slit illumination beams as well. The movement of the slit illuminator 106 can be controlled by a controller 112.

As shown in this specific example, the controller 112 is mounted to the frame 102 and is communicatively coupled to the slit illuminator 106. However, in some other embodiments, the controller 112 may not be mounted to the frame 102. Indeed, the controller 112 may be remote from the frame 102. During examination, the controller 112 controls the slit illuminator 106 to illuminate the eye 10 of the patient 12 with one or more slit illumination beams either sequentially or simultaneously.

Still referring to FIG. 1, the camera 108 generate one or more images of the eye 10 during the illumination by the slit illuminator 106. The camera 108 is communicatively coupled to the controller 112. In some embodiments, the image(s) can be communicated to a network or stored on a memory for later consultation by trained optometrists, ophthalmologist and other eye care professionals. In some embodiments, the camera 108 can be any suitable type of camera such as a charged-coupled device (CCD) camera 120, which is configured to generate images of the illuminated eye 10. For instance, the CCD camera 120 can have a resolution higher than 2 megapixels, preferably above 4 megapixels and most preferably above 8 megapixels. The camera 108 can be part of a mobile device in some embodiments. For instance, the type of mobile device can include, but is not limited to, a smartphone such as the iPhone® (any generation), the Android® phone (any generation) and the like, an electronic tablet such as the iPad® (any generation, the Android® tablet (any generation) and the like.

In some embodiments, the camera can be a two-dimensional camera. In some embodiments, the camera 108 can be a three-dimensional (3D) camera so as to generate 3D images of the so-illuminated eye. For instance, the 3D camera can be a stereoscopic camera in some embodiments as the 3D camera can be a light field camera (also referred to as “plenoptic camera” in the field) in some other embodiments. An example of such light field camera is manufactured by Raytrix GmbH, Germany.

In some embodiments, it is envisaged that the camera 108 can be movable relative to the face of the patient 12. For instance, the camera 108 may be mechanically connected to the frame 102 via an articulated arm, or any other suitable type of actuator. In such embodiments, the camera 108 can be moved in any given coordinate system x, y, z relative to the eye of the patient. Accordingly, by moving the camera 108 during examination, one or more images of the eye of the patient can be taken from different spatial positions while the eye 10 is being illuminated by one or more of the illumination patterns or beams. The movement of the camera 108 can be controlled by the controller 112.

As best shown in FIG. 1A, the slit illuminator 106 can be configured to illuminate the eye 10 of the patient 12 from a first viewpoint A. In other words, the slit illuminator 106 is configured to shine a slit illumination beam 110 along a given illumination path 114. In some embodiments, the first viewpoint A forms an incident angle ω with a sagittal plane 116 of the head of the patient 12. Preferably, the first viewpoint A or illumination path 114 is non-perpendicular to an iris 10a of the patient's eye 10. In the illustrated embodiment, the camera 108 has a second viewpoint B different from the first viewpoint A. Accordingly, the camera 108 has an imaging path 118 which originates from the eye 10 of the patient 12 and leads to the camera 108. The first and second viewpoints A and B, and therefore the illumination and imaging paths 114 and 118, differ from one another. As depicted, the illumination and imaging paths 114 and 118 are non-parallel to one another. It is noted that the illumination and imaging paths 114 and 118 are oversimplified in FIG. 1A for ease of understanding. It is intended that the illumination and imaging paths 114 and 118 can be more complex in some other embodiments. As such, the slit illuminator 106 can include a variety of other optical components, such as shutter(s), mirror(s), diffuser(s), filter(s) and the like to propagate, carry and/or modify the light generated by the slit illuminator 106a and the background illuminator 106b.

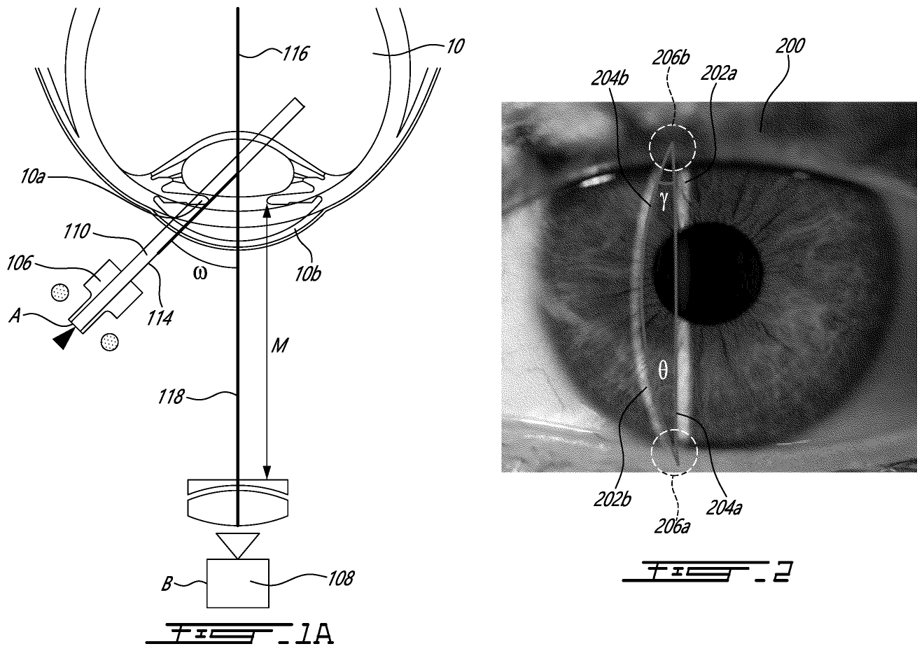

It is noted that the slit illuminator 106 and the camera 108 have different viewpoints A and B relative to the patient's eye 10. Accordingly, when the eye 10 is illuminated from the first viewpoint A, imaging from the second viewpoint results in a first image 200 showing a first line element 202a indicative of a reflection of the slit illumination beam on the iris 10a of the patient's eye 10 and a second line element 202b indicative of a reflection of the first slit illumination beam within a cornea 10b of the patient's eye, an example of which is shown at FIG. 2. The first and second line elements 202a and 202b are discernable by an increased brightness compared to the remainder of the eye as imaged. As described in further detail below, the system described herein is configured for evaluating an angle value indicative of an angle formed between the first and second line elements 202a and 202b of the first image 200. More specifically, the controller is configured for fitting first and second lines 204a and 204b to a respective one of the first and second line elements 202a and 202b in the first image 200. After the first and second lines 204a and 204b have been fitted, the controller is configured to identify a first intersection 206a of the first and second lines 204a and 204b. Then, the controller is configured to determine a first angle value θ indicative of an angle formed between the first and second lines 204a and 204b at the first intersection 206a. As the first angle value θ is somewhat associated to a depth of the anterior chamber of the eye 10, or to the iridocorneal angle of the eye 10, the condition of the patient's eye can be assessed based on the first angle value θ. Using reference data associating reference angle values to corresponding reference eye conditions, the first angle value θ can then be associated to an iridocorneal angle of the patient's eye, or directly to a condition such as glaucoma, or any other angle-relate eye condition. As discussed herein, in some other embodiments, the controller determines a second angle value γ indicative of an angle formed between the first and second lines 204a and 204b at the second intersection 206b. The assessment of the condition of the eye can factor in either one or both of the first and second angle values θ and γ.

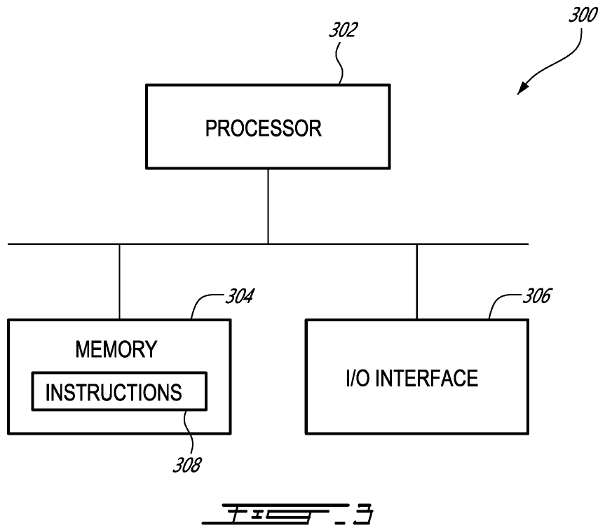

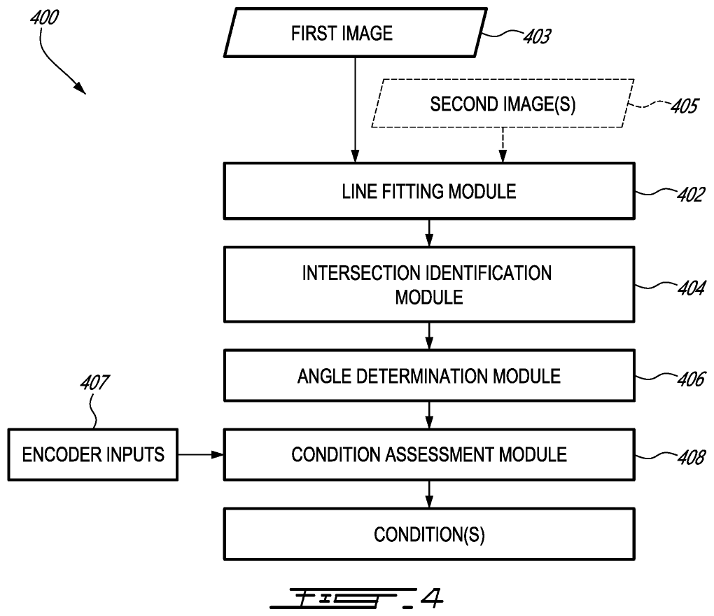

The controller 112 can be provided as a combination of hardware and software components. The hardware components can be implemented in the form of a computing device 300, an example of which is described with reference to FIG. 3. Moreover, the software components of the controller 112 can be implemented in the form of a software application, an example of which is described with reference to FIG. 4.

Referring to FIG. 3, the computing device 300 can have a processor 302, a memory 304, and I/O interface 306. Instructions 308 for assessing the condition of the eye 10 of the patient 12 based on the image(s) can be stored on the memory 304 and accessible by the processor 302.

The processor 302 can be, for example, a general-purpose microprocessor or microcontroller, a digital signal processing (DSP) processor, an integrated circuit, a field-programmable gate array (FPGA), a reconfigurable processor, a programmable read-only memory (PROM), or any combination thereof.

The memory 304 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 306 enables the computing device 300 to interconnect with one or more input devices, such as mouse(s), keyboard(s), camera(s), face sensor(s), or with one or more output devices such as display(s), network(s), memory (ies).

Each I/O interface 306 enables the controller 112 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Referring now to FIG. 4, the software application 400 is configured to assess the condition of the eye based on the image(s) captured by the camera 108 while the patient's eye 10 is being illuminated by the slip lamp unit. In some embodiments, the software application 400 is stored on the memory 304 and accessible by the processor 302 of the computing device 300.

As shown, the software application 400 has a number of modules communicating with each other. Each of the modules has a software portion and a hardware portion which work together to receive the image(s), process the image(s) and output qualitative information carried by the image(s). As depicted, the software application 400 has a line fitting module 402, an intersection identification module 404, an angle determination module 406 and a condition assessment module 408. The line fitting module 402 receives a first image 403 from the camera or other computer-readable memory. The line fitting module 402 finds first and second line elements in the first image 403. The first line element is an elongated region of enhanced brightness compared to the remainder of the eye and shows a reflection of the slit illumination beam onto the iris of the patient's eye. The second line element is an elongated region of enhanced brightness compared to the remainder of the eye, and in some embodiments of lower brightness than that of the first line element, and shows a reflection of the slit illumination beam within the cornea of the patient's eye. Both the first and second line elements have their corresponding thickness and brightness which can be recognized and identified by the line fitting module 402. As such, the line fitting module 402 fits first and second lines to the first and second line elements in the first image, respectively. Depending on the embodiment, the first and second lines can follow an interior boundary of the first and second line elements, an outer boundary of the first and second line elements, or a middle line of the first and second line elements. Typically, the first and second lines are continuous and smooth lines, with the first line being often times less curved than the second line. The first and second lines can be expressed in terms of mathematical equations y varying as a function of x, where x and y are x- and y-Cartesian coordinates of the image, for instance. The intersection identification module 404 receives the first and second lines and calculates the position(s) where the first and second lines meet. There can be one or two of such intersections. In embodiments where the first and second lines meet twice, for instance at a lower portion of the eye and at an upper portion of the eye, the intersection identification module 404 can identify and position these two intersections. This information is communicated to the angle determination module 406 which, based on the first and second lines, determine an angle value (or two angle values) for the intersection(s) identified above. Once the angle values have been determined, the condition assessment module can assess a condition of the eye-based thereon.

In some embodiments, a second image 405 of the same eye can be fed to the software application 400. In these embodiments, the second image 405 can be an image acquired simultaneously or sequentially to the capture of the first image 403. The second image 405 can differ from the first image 403 in many ways including, but not limited to, different illumination in terms of slit thickness, slit orientation and the like, captured when the patient's eye is illuminated from different viewpoints, captured with a camera (or cameras) having different viewpoints, and the like. It is noted that the condition assessment can be enhanced when using more than one angle value. For instance, a first angle value of a first intersection in the first image can be used and provide satisfactory condition assessment. However, in some other embodiments, determining a second angle of a second intersection in the first image can further help condition assessment. Moreover, if a second image is captured, then additional second angle value(s) (one for each intersection) can contribute to the condition assessment.

In some embodiments, a first encoding device monitors the first viewpoint of the slit illuminator with respect to the patient's eye when the first and second images are generated, and generating a signal indicative of the first viewpoint. As such, the first encoding device can monitor an orientation of the slit illumination beam with respect to the frame of the slit illuminator, and then generate a signal indicative of an orientation angle of the slit illumination beam with respect to the patient's eye and/or to the slit illuminator when the image(s) are generated. In some embodiments, the orientation angle is associated to each of the image(s) generated. Further, a second encoding device can monitor the second viewpoint of the camera with respect to the patient's eye when the first and second images are generated. Moreover, a third encoding device can monitor an orientation of the slit illumination beam with respect to the frame when the first and second images are generated. Accordingly, in some embodiments, the first viewpoint, the second viewpoint and/or the orientation of the slit illumination beam associated to each image, or any other encoder inputs 407, can be received at the condition assessment module 408. As such, the condition assessment module 408 can rely not only on the first and second angle value(s) associated to the first and second images, but also on the known configuration of the system when the first and second images were generated.

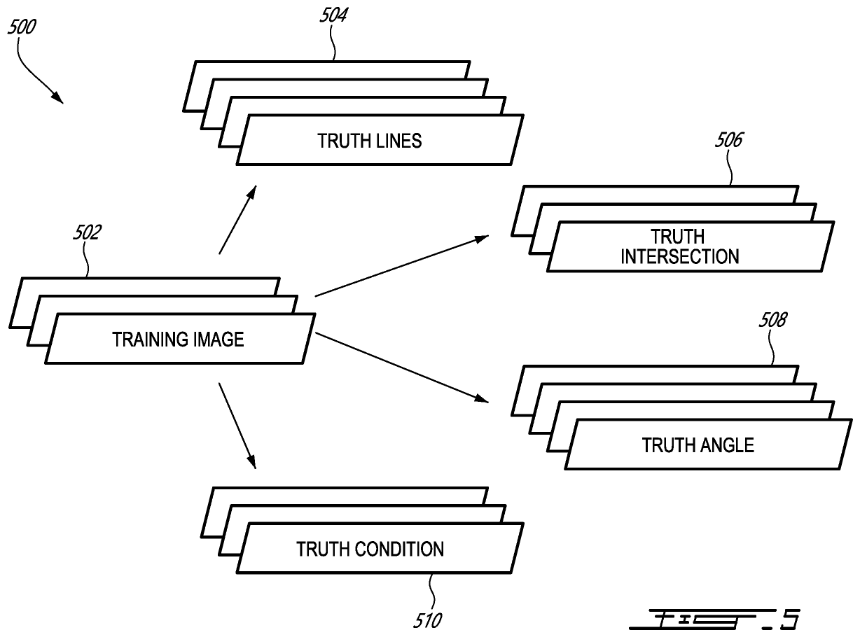

Referring now to FIG. 5, it is encompassed that any one of the modules 402 to 408 can be trained using artificial intelligence algorithms. Accordingly, such trained engine(s) can perform the fitting, identifying, determining and/or assessing performed by modules 402-408. In some embodiments, the line fitting module 402 has been trained using supervised learning during which the line fitting module 402 is trained to identify lines associated to particular line elements in a set of training images 502 each showing first and second line elements and having truth lines 504 associated therewith. In some embodiments, the intersection identification module 404 has been trained using supervised learning during which the intersection identification module 404 is trained to identify intersection(s) associated to particular lines in a set of training images 502 each showing first and second line elements, and corresponding intersections, and having truth intersections 506 associated therewith. In some embodiments, the angle determination module 406 has been trained using supervised learning during which the angle determination module 406 is trained to identify angle(s) of line intersection(s) in a set of training images 502 each showing first and second line elements and intersections, and having truth angles 508 associated to each of the training images 502. In some embodiments, the truth assessment module 408 has been trained using supervised learning during which the truth assessment module 408 is trained to assess condition(s) of the eye in a set of training images 502 each showing the determined angle(s), and having truth conditions 510 associated to each of the training images 502. For instance, the truth data can be provided in the form of a training image which has been annotated (e.g., colored) to show the line(s), intersection(s), angle(s) and conditions(s) shown in the corresponding training image.

Indeed, as mentioned above, the trained engines 402-408 are trained using supervised learning. In such supervised learning, each training image in the set of training images may be associated with a label while training. Supervised machine learning engines can be based on Artificial Neural Networks (ANN), Support Vector Machines (SVM), capsule-based networks, Linear Discriminant Analysis (LDA), classification tree, a combination thereof, and any other suitable supervised machine learning engine. However, as can be understood, in some other embodiments, it is intended that the trained engines 402-408 can be trained using unsupervised where only training images are provided (no desired or truth outputs are given), so as to leave the trained engines 402-408 find a structure or resemblance in the provided training images. For instance, unsupervised clustering algorithms can be used. Additionally or alternately, the trained engines 402-408 can involve reinforcement learning where the trained engines 402-408 interact with example training images and when they reach desired or truth outputs, the trained engines 402-408 are provided feedback in terms of rewards or punishments. Two exemplary methods for improving classifier performance include boosting and bagging which involve using several classifiers together to “vote” for a final decision. Combination rules can include voting, decision trees, and linear and nonlinear combinations of classifier outputs. These approaches can also provide the ability to control the tradeoff between precision and accuracy through changes in weights or thresholds. These methods can lend themselves to extension to large numbers of localized features. In any case, some of these engines may involve human interaction during training, or to initiate the engine, however human interaction may not be involved while the engine is being carried out, e.g., during analysis of an accessed image. See Nasrabadi, Nasser M. “Pattern recognition and machine learning.” Journal of electronic imaging 16.4 (2007): 049901 for further detail concerning such trained engines.

The computing device 300 and the software application 400 described above are meant to be examples only. Other suitable embodiments of the controller 112 can also be provided, as it will be apparent to the skilled reader.

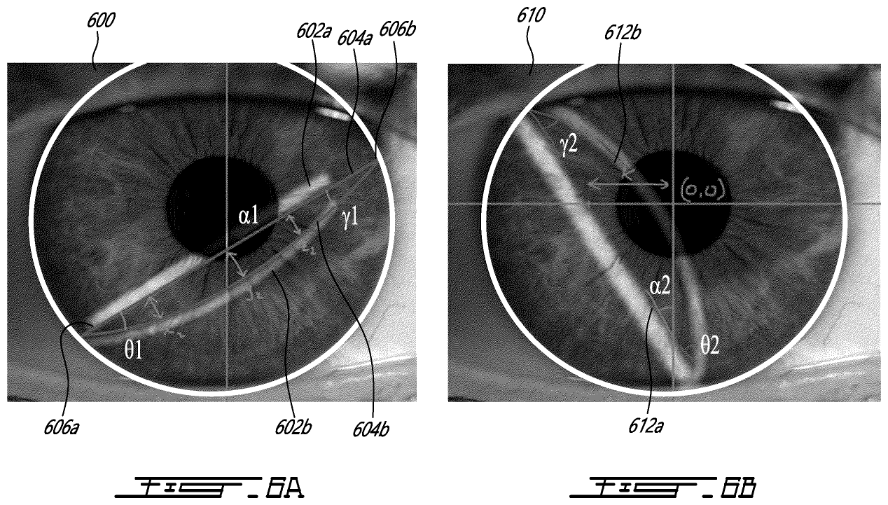

In some embodiments, the system can be used to image the patient's eye with different illumination patterns, an example of which is described in FIGS. 6A and 6B. As shown, FIG. 6A shows a first image 600 of a patient's eye being illuminated with a first slit illumination beam having a first orientation α1 relative to the vertical. The first and second line elements 602a and 602b are thereby oriented with a similar angle relative to the vertical. As shown, first and second lines 604a and 604b identified by the line fitting module. The first and second lines 604a and 604b are overlaid on the first image 600 for clarity. In this embodiment, the first and second lines 604a and 604b are within an inner boundary of the first and second line elements 602a and 602b, respectively. As shown, first and second intersections 606a and 606b are identified where the first and second lines 602a and 602b intersect with one another, and first and second angle values θ1 and γ1 are determined on that basis. In some embodiments, the condition of the patient's eye is assessed on the basis not only of a single one of the first and second angle values θ1 and γ1, but based on both the first and second angle values θ1 and γ1. In some embodiments, a pixel count indicative of a number of pixels extending between the first and second lines 602a and 602b is determined. In these embodiments, the pixel count can be used in the condition assessment. A greater pixel count being indicative of a larger volume between the iris and the cornea of the patient's eye, in some embodiments. The first and second angle values θ1 and γ1 can be used to create a cornea model and an iris model in some embodiments. In these embodiments, whether a pixel is considered between the first and second lines 602a and 602b can depend on the proportion of the pixel which is inside the first and second lines 602a and 602b. In some embodiments, when more than 50 percent of the pixel (area-wise) is within the first and second lines 602a and 602b, the pixel is counted. Otherwise, the pixel can be omitted.

FIG. 6B shows a second image 610 of the patient's eye being illuminated with a second slit illumination beam having a second orientation α2 relative to the vertical, with the first and second orientations α1 and α2 being different. In this embodiment, the second image 610 has been captured shortly after the capture of the first image 600. Accordingly, the first and second images 600 and 610 show the patient's in similar conditions. The second image is processed to fit first and second lines 612a and 612b, identify first and second intersections of these lines 612a and 612b, and determine first and second angles values θ2 and γ2. In some embodiments, the condition of the patient's eye is assessed based on the first and second angle values θ1 and γ1 of the first image 600 and also based on the first and second angle values θ2 and γ2 of the second image 610. In some embodiments, the first and second angle values θ2 and γ2 of the second image 610 can be used to complement the corneal model and the iris model created on the basis of the first image 600.

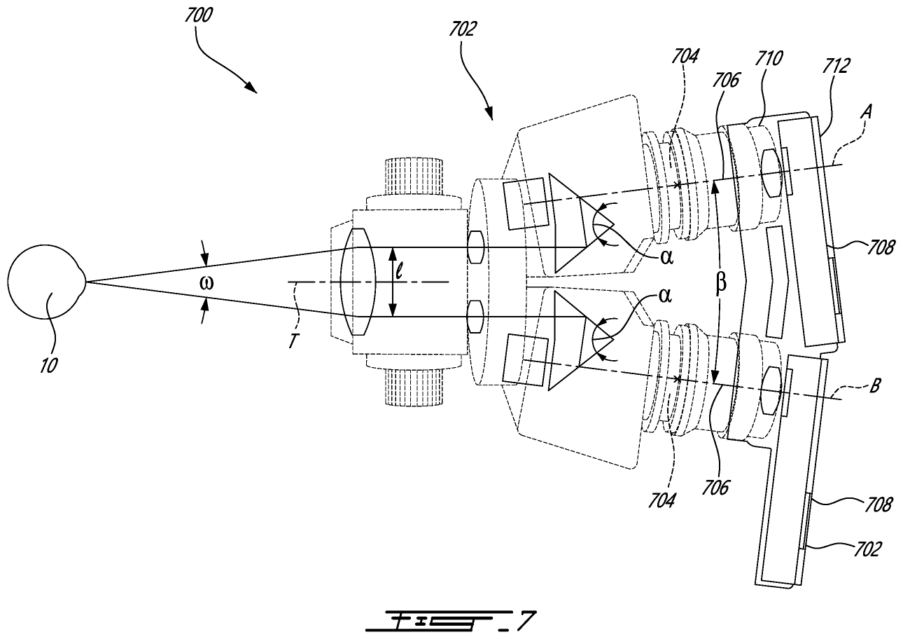

FIG. 7 shows an example of a system 700 of the binocular type. As depicted, the system 700 has a binocular scope 702 optically coupled to two ocular elements 704 which are transversally spaced apart from one another. In this disclosure, the transverse orientation T is generally perpendicular to a sagittal plane of the system 700 and to the vertical orientation. The binocular scope 702 and the two ocular elements 704 collectively form first and second images of the eye 10 propagating along corresponding eye imaging paths 706, with each ocular elements forming a corresponding image at an imaging plane axially spaced apart along axes A and B of the ocular elements 704 during the imaging process. As shown, the system 700 can include a variety of other optical components, such as shutter(s), mirror(s), diffuser(s), filter(s) and the like to propagate, carry and/or modify the light incoming from the eye 10 of the patient. As shown, the system 700 has a mounting bracket 710 to be removably attached to the system 100, and more specifically to the ocular elements 704 in this specific embodiment. As shown, the mounting bracket 710 has two transversally spaced-apart camera receivers 712 which are each configured to removably receive a corresponding one of a pair of cameras 708. As shown, the cameras 708 can be part of corresponding mobile devices. As such, in some embodiments, the camera receivers 712 can be provided in the form of mobile device receivers. The mounting bracket 710 also has two transversally spaced-apart camera through apertures extending through the mounting bracket 710. Using the system 700, first and second images of the patient's eye can be captured simultaneously, but from different viewpoints A and B.

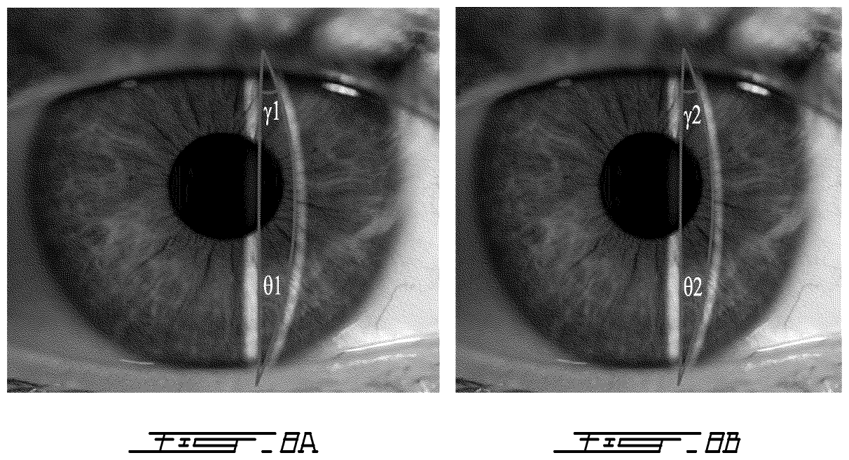

FIGS. 8A and 8B show examples of first and second images captured simultaneously by the cameras of the system 700. Since the cameras are spaced apart from one another, the cameras have different viewpoints relative to the patient's eye. Accordingly, the first and second images carry slightly different information, which can be used to enhance the condition assessment performed by the system.

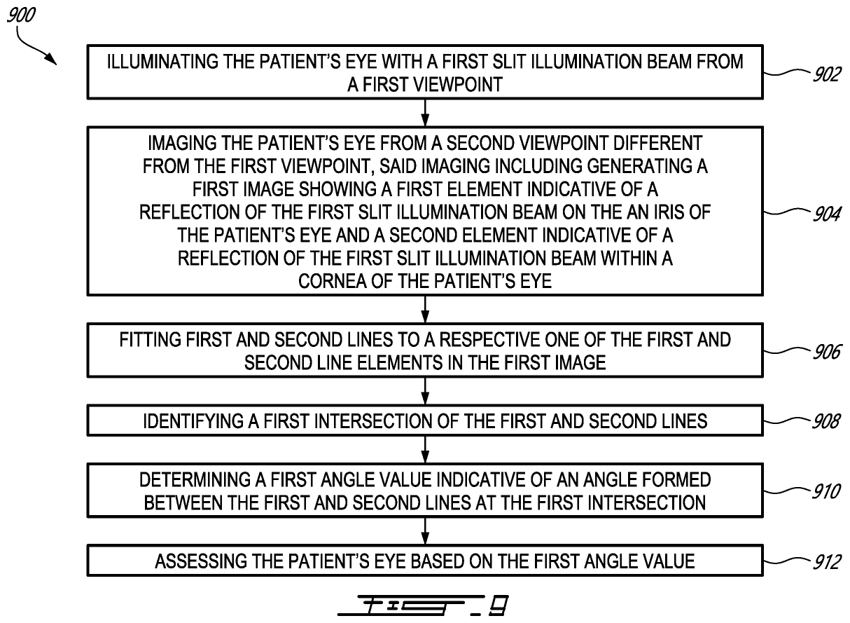

FIG. 9 shows a flow chart of an example of a method 900 of assessing a condition of a patient's eye. The method 900 can be performed using the system(s) described above with reference to FIGS. 1 and 7, for instance.

At step 902, the patient's eye is illuminated with a first slit illumination beam from a first viewpoint. At step 904, the patient's eye is imaged during the illumination of step 902 from a second viewpoint, with the second viewpoint being different from the first viewpoint. The step 904 of imaging includes generating a first image showing a first line element indicative of a reflection of the first slit illumination beam on an iris of the patient's eye and a second line element indicative of a reflection of the first slit illumination beam within a cornea of the patient's eye. At step 906, first and second lines are fitted to a respective one of the first and second line elements in the first image. In some embodiments, the first line and the first line element are one of curved and linear. The second line and the second line element are straight lines. At step 908, a first intersection of the first and second lines is identified in the first image. At step 910, a first angle value indicative of an angle formed between the first and second lines at the first intersection is determined. At step 912, the condition of the patient's eye is assessed based at least on the first angle value.

In some embodiments, the step 912 can include a step of matching the first angle value to the condition of the eye based on reference data associating reference angle values to corresponding reference eye conditions. In these embodiments, the reference angle values can originate from reference measurements performed at the first and second viewpoints. Accordingly, different first and second viewpoints can lead to different reference data.

In some embodiments, the method 900 can include steps for determining second angle values indicative of another angle formed in the first image and/or a second image, as discussed above. The second image can be captured sequentially or simultaneously to the first image. In some embodiments, the second image is captured from a viewpoint that is different from a viewpoint of the first image. In some embodiments, the second image is captured when the slit illumination beam has a given width, given position, and/or given orientation relative to the patient's eye being different than those of the first slit illumination beam used to illuminate the patient's eye during the capture of the first image. In any case, the step 912 of assessing can be further based on the second angle value(s) that can be measured in the first image or in additional second images. In some embodiments, the method 900 includes a step of generating iris and cornea 2D or 3D models based on the first and second angle values. It is intended that the iris and cornea 2D or 3D models can be displayed on a display screen, communicated to an external server or network and/or stored onto a computer-readable memory. In these embodiments, the iris and cornea 2D or 3D models can be associated to an identification number or name of the patient, a date, an assessed condition and the like. In some embodiments, the method 900 can include a step of determining a thickness of the cornea across the section of the cornea that is illuminated by the slit illumination beam. The thickness of the cornea can be inferred from a thickness of the second line element, for instance. In some embodiments, different images taken with slit illumination beams incoming from different viewpoints or different orientation angles can allow the reconstruction of a cornea model being informative of a thickness of the cornea at a plurality of locations.

As can be understood, the examples described above and illustrated are intended to be exemplary only. For instance, the slit illuminators and cameras can have a fixed position relative to a frame with the advantage of being able to capture images simultaneously from different viewpoints and potentially omitting a movement mechanism. It will be understood that the method and system can be used to scan or otherwise acquire the thickness of the entire cornea in some embodiments. It is understood that the slit illuminator can be provided in one of many forms including, but not limited to, a slit lamp unit, a slit projector, a slit laser projector (having a laser beam of an eye-safe wavelength such as an infrared wavelength, which can conveniently maintain the iris dilated during illumination, but require an infrared camera), a grid-based slit illuminator, and the like. In some embodiments, the method and systems involve a plurality of slit illuminators, and/or a plurality of cameras, each having respective fixed or movable viewpoints relative to the patient's eye. The scope is indicated by the appended claims.

What is claimed is:

1. A method of assessing a condition of a patient's eye, the method comprising:
using a slit illuminator, illuminating the patient's eye with a first slit illumination beam from a first viewpoint;
using a camera and during said illuminating, imaging the patient's eye from a second viewpoint different from the first viewpoint, said imaging including generating a first image showing a first line element indicative of a reflection of the first slit illumination beam on an iris of the patient's eye and a second line element indicative of a reflection of the first slit illumination beam within a cornea of the patient's eye; and
using a controller, fitting first and second lines to a respective one of the first and second line elements in the first image, identifying a first intersection of the first and second lines; determining a first angle value indicative of an angle formed between the first and second lines at the first intersection; and assessing the condition of the patient's eye based on the first angle value.

2. The method of claim 1 wherein the first slit illumination beam is directly focused on the patient's eye.

3. The method of claim 1 wherein the first line is one of curved and linear, and the second line is linear.

4. The method of claim 1 further comprising identifying a second intersection of the first and second lines different from the first intersection and determining a second angle value indicative of an angle formed between the first and second lines at the second intersection.

5. The method of claim 1 further comprising illuminating the patient's eye with a second slit illumination beam having a third viewpoint different from the first viewpoint, and imaging the patient's eye during said illuminating with the second slit illumination beam, said imaging generating a second image showing a third line element indicative of a reflection of the second slit illumination beam on the iris of the patient's eye and a fourth line element indicative of a reflection of the second slit illumination beam within the cornea of the patient's eye, the method further comprising repeating said fitting, said identifying and said determining for said second image, thereby outputting a second angle value on which said assessing is further based.

6. The method of claim 1 wherein the first slit illumination beam has a first orientation with respect to the slit illuminator, the method further comprising illuminating the patient's eye with a second slit illumination beam having a second orientation being different from the first orientation, and imaging the patient's eye during said illuminating with the second slit illumination beam, said imaging generating a second image showing a third line element indicative of a reflection of the second slit illumination beam on the iris of the patient's eye and a fourth line element indicative of a reflection of the second slit illumination beam within the cornea of the patient's eye, the method further comprising repeating said fitting, said identifying and said determining for said second image, thereby outputting a second angle value on which said assessing is further based.

7. The method of claim 1 further comprising determining a thickness of the cornea based on a thickness of the second line element.

8. The method of claim 1 further comprising generating at least one of an iris three-dimensional (3D) model and a cornea 3D model based at least on the first angle value.

9. The method of claim 1 wherein said assessing includes matching the first angle value to the condition of the patient's eye based on reference data associating reference angle values to corresponding reference eye conditions.

10. The method of claim 9 wherein the reference angle values originate from reference measurements performed at the first and second viewpoints.

11. The method of claim 1 further comprising determining a pixel count indicative of a number of pixels extending between the first and second lines, wherein said assessing is further based on said pixel count.

12. The method of claim 11 wherein said controller has a trained engine performing at least one of said fitting, said identifying, said determining and said assessing.

13. A system for assessing a condition of a patient's eye, the system comprising:
a frame;
a slit illuminator mounted to the frame and having a first viewpoint, the slit illuminator being configured for illuminating the patient's eye with a slit illumination beam;
a camera mounted to the frame and having a second viewpoint different from the first viewpoint, the camera being configured for imaging the patient's eye during said illuminating, said camera generating a first image showing a first line element indicative of a reflection of the slit illumination beam on an iris of the patient's eye and a second line element indicative of a reflection of the slit illumination beam within a cornea of the patient's eye; and
a controller communicatively coupled to the camera, the controller having a processor and a memory having stored thereon instructions that when executed by the processor perform the steps of: fitting first and second lines to a respective one of the first and second line elements in the first image, identifying a first intersection of the first and second lines; determining a first angle value indicative of an angle formed between the first and second lines at the first intersection; and assessing the condition of the patient's eye based on the first angle value.

14. The system of claim 13 wherein the slit illuminator is movably mounted to the frame via a first encoding device, the first encoding device monitoring the first viewpoint of the slit illuminator with respect to the patient's eye when said first image is generated, and generating a signal indicative of the first viewpoint.

15. The system of claim 14 wherein the first encoding device is further configured for monitoring an orientation of the slit illumination beam with respect to the frame, and generating a signal indicative of an orientation angle of the slit illumination beam when said first image is generated.

16. The system of claim 13 wherein the camera is movably mounted to the frame via a second encoding device, the second encoding device monitoring the second viewpoint of the camera with respect to the patient's eye when said first image is generated, and generating a signal indicative of the second viewpoint.

17. The system of claim 13 wherein said assessing includes matching the first angle value to the condition of the patient's eye based on reference data associating reference angle values to corresponding reference eye conditions.

18. The system of claim 17 wherein the reference angle values originate from reference measurements performed at the first and second viewpoints.

19. The system of claim 13 further comprising determining a pixel count indicative of a number of pixels extending between the first and second lines, wherein said assessing is further based on said pixel count.

20. The system of claim 13 wherein said controller has a trained engine performing at least one of said identifying, determining and assessing.
